(19) United States
(12) Patent Application Publication
(10) Pub. No.: US 2004/0228871 A1
(43) Pub. Date: Nov. 18, 2004

(54) Treatment and analysis of proliferative disorders

(76) Inventors: Tayyaba Hasan, Boston, MA (US); Bernard Ortel, Boston, MA (US)

Correspondence Address: FROMMER LAWRENCE & HAUG, 745 FIFTH AVENUE- 10TH FL., NEW YORK, NY, 10151, US

(21) Appl. No.: 10/818,906

(22) Filed: Apr. 5, 2004

Related U.S. Application Data
(63) Continuation-in-part of application No. 09/586,235, filed on Jun. 2, 2000.
(60) Provisional application No. 60/137,365, filed on Jun. 3, 1999.
(60) Provisional application No. 60/460,497, filed on Apr. 4, 2003.

(51) Int. Cl. A61K 39/395; A61K 31/555

(52) U.S. Cl. 424/178.1; 514/185; 514/410

(57) ABSTRACT

A method of treating a subject having unwanted cell proliferation which includes: inducing differentiation in a cell; providing said cell with a photosensitizer; and activating said PS, thereby the treating unwanted cell proliferation.

### CROSS-REFERENCE TO RELATED APPLICATIONS

This application is a continuation-in-part of U.S. application Ser. No. 09/586,235, filed on Jun. 2, 2000, claiming priority to U.S. Provisional Application Serial No. 60/137,365, filed Jun. 3, 1999, the contents of which are incorporated herein by reference, including the prosecution history of U.S. application Ser. No. 09/586,235. This application also claims priority to U.S. Provisional Application Serial No. 60/460,497, filed on Apr. 4, 2003, the contents of which are incorporated herein by reference.

### STATEMENT OF POTENTIAL GOVERNMENT INTEREST

This work was funded by a National Institutes of Health grant no. NIH5R01 AR40352B06 and a grant by the Massachusetts Department of Public Health, 34081126112.

Each of the reference and documents (including any manufacturer's specifications, instructions, etc.) is hereby expressly incorporated herein by reference. Furthermore, all documents cited in this text, and all documents cited or referenced in documents cited in this text, and any manufacturer's instructions or catalogues for any products cited or mentioned in this text or in any document hereby incorporated into this text, are hereby incorporated herein by reference. Documents incorporated by reference into this text or any teachings therein can be used in the practice of this invention. Documents incorporated by reference into this text are not admitted to be prior art. Furthermore, authors or inventors on documents incorporated by reference into this text are not considered to be “another” or “others” as to the present inventive entity and vice versa, especially where one or more authors or inventors on documents incorporated by reference into this text are an inventor or inventors named in the present inventive entity.

### FIELD OF THE INVENTION

The invention relates to the integration of photodynamic therapy (PDT) and differentiation therapy (DT) in the inhibition of unwanted cell proliferation, and in the analysis, e.g., diagnosis or staging of a disorder.

### BACKGROUND OF THE INVENTION

Photodynamic therapy (PDT) can be defined as the combined action of a photoactivable compound (any photosensitizer, PS) and visible electromagnetic radiation in the presence of oxygen (Hasan et al., (eds.), Cancer Medicine, 4th edition, pp. 739-751. Baltimore: Williams & Wilkins, 1996.). PDT can be used to treat neoplastic and some other proliferative diseases. The advantage over other forms of cancer therapies lies in the dual selectivity of PDT, which is due to (1) a preferential accumulation and/or retention of the PS in malignant tissue, and (2) the possibility of localized light delivery. Under optimal conditions, neither light nor PS will cause toxicity by themselves. This again spares normal tissue and prevents systemic toxicity. Besides its application in PDT, the preference of certain PS for malignant cells and/or tissue may be used for diagnostic purposes by exploiting the fluorescent properties of the photoactivable compounds. Several reviews in the recent past have summarized the principles, applications and ongoing research in this field (Henderson et al., Photochem. Photobiol. 55: 145-157, 1992; Fisher et al., Lasers Surg.Med. 17: 2-31, 1995; and Dougherty et al., Photodynamic therapy. J. Natl Cancer Instit. 90: 889-905, 1998).

Most clinical experience has been obtained with hematoporphyrin derivative (HpD). HpD-derived sensitizers such as Photofrin™ are at this time most widely approved internationally. However, its poor chemical definition, the prolonged cutaneous photosensitivity associated with systemic HpD administration, and the suboptimal wavelength range of excitation (630 nm instead of >700 nm) have led to interest in other PS's. More recently, the induction of protoporphyrin IX (PPIX), a photosensitizer synthesized intracellularly by exposure of cells or tissue to exogenous ALA, has been introduced into the PDT arena for potential use as a diagnostic and photosensitizing strategy (Peng et al., Cancer. 79: 2282-308, 1997).

### SUMMARY OF THE INVENTION

The invention is based, in part, on the discovery that the integration of elements of photodynamic therapy (PDT) and differentiation therapy (DT) enhances the inhibition of unwanted cell proliferation, and can be used for the analysis, e.g., the diagnosis or staging, of unwanted cell proliferation.

Accordingly, the invention features a method of treating a cell of a subject, e.g., a subject having a disorder characterized by unwanted cell proliferation, e.g., cancer. The method includes: inducing differentiation in a cell, e.g., by administration of an exogenous agent; providing said cell with a photosensitizer (PS); and activating said PS, thereby treating unwanted cell proliferation.

In a preferred embodiment, a PS or the metabolic precursor of a PS and a differentiating agent are administered to the subject, or are applied to a sample of the subject.

In a preferred embodiment, the subject is a patient having a malignant or benign disorder characterized by unwanted cell proliferation.

In a preferred embodiment, a compound, e.g., ALA or ALA esters, which causes the accumulation of a PS, the formation of a PS, or is converted to a PS, in the subject's body, or in a sample of the subject, is administered to the subject, or administered to the sample taken from the subject. For example, a compound which causes the accumulation of, the formation of, or which is converted to, a porphyrin, or a porphyrin precursor, is administered to the subject, or to a sample taken from the subject.

In preferred embodiments: the photosensitizer has a chemical structure that includes multiple conjugated rings that allow for light absorption and photoactivation, e.g., the photosensitizer can produce singlet oxygen upon absorption of electromagnetic irradiation at the proper energy level and wavelength; the photosensitizer can include a porphyrin, porphyrin derivative or analog thereof, e.g., a tetrapyrroles; or the photosensitizer can include chlorin e6, a chlorin derivative or analog thereof. Suitable photosensitizers include photofrin®; synthetic diporphyrins and dichlorins; hydroporphyrins, e.g., chlorins and bacteriochlorins of the tetra(hydroxyphenyl) porphyrin series; phthalocyanines; o-substituted tetraphenyl porphyrins (picket fence porphyrins); 3,1-meso tetrakis (o-propionamido phenyl) porphyrin; Verdins; Purpurins, e.g., tin and zinc derivatives of octaethylpurpurin (NT2), and etiopurpurin (ET2); Chlorins, e.g., chlorin e6, and mono-1-aspartyl derivative of chlorin e6; Benzoporphyrin derivatives (BPD), e.g., benzoporphyrin monoacid derivatives, tetracyanoethylene adducts of benzoporphyrin, dimethyl acetylenedicarboxylate adducts of benzoporphyrin, Diels-Adler adducts, and monoacid ring “A” derivative of benzoporphyrin; Low density lipoprotein mediated localization parameters similar to those observed with hematoporphyrin derivative (HPD); sulfonated aluminum phthalocyanine (Pc), sulfonated AlPc disulfonated (AIPcS2), tetrasulfonated derivative, sulfonated aluminum naphthalocyanines, chloroaluminum sulfonated phthalocyanine (CASP); zinc naphthalocyanines; anthracenediones; anthrapyrazoles; aminoanthraquinone; phenoxazine dyes; phenothiazine derivatives; chalcogenapyrylium dyes cationic selena and tellurapyrylium derivatives; ring-substituted cationic PC; pheophorbide; hematoporphyrin (HP); protoporphyrin; ALA; and ALA-esters, hexyl ester or methyl ester.

In a preferred embodiment, the PS is coupled to a targeting moiety. Examples of targeting moieties include antibodies, or antigen binding fragments thereof, and ligands for cell surface molecules.

In one embodiment, the differentiating agent and PS are administered individually, or as a combination. In another embodiment, the differentiating agent is administered before administration of PS. In another embodiment, the differentiating agent is administered after administration of PS.

In a preferred embodiment, the subject has a hematopoietic disorder, e.g., leukemia, e.g., acute promyelocytic leukemia. Preferred treatment includes administration of a PS and a differentiating agent, e.g., retinoic acid, to the subject.

In a preferred embodiment, the subject has a malignancy of the epithelium, e.g., of breast epithelial cells. Preferred treatment includes administration of a PS and an antidiabetic compound, e.g., troglituzone, or a ligand for a transcription factor, e.g., transcription factor PPAR gamma, is administered to the subject.

In a preferred embodiment, the subject has an unwanted proliferation of prostate cells, e.g., prostate carcinoma. Preferred treatment includes administering a PS and a hormonal agent, e.g., a retinoid or vitamin D, or an agent which increases levels of retinoic acid, e.g., liarozole or Liazal, to the subject.

In a preferred embodiment, a tumor cell is induced to differentiate and the PS is supplied such that it is present while the cells are in a state of induced differentiation. This timing is particularly preferred when using DT agents that only temporarily induce differentiation. The activating light can also be supplied while the cell is in the induced state.

In a preferred embodiment, the cell is an: epithelial cell; prostate cell; a nerve cell, or blood cell, e.g., a white blood cell.

In a preferred embodiment, the cell is other than a cell that secretes interferon-gamma (IFN-gamma) or epidermal growth factor-alpha (EGF-alpha), or other than a cell that is activated or differentiating due to contact with interferon-gamma (IFN-gamma) or epidermal growth factor-alpha (EGF-alpha). For example, the cell is not a differentiating skin cell, e.g., an epidermal keratinocyte that has psoriasis Vulgaris.

In another preferred embodiment, a cell that is differentiating (e.g., due to the action of an endogenous agent in the subject, e.g., the action of a cytokine) is contacted with an exogenous differentiating agent, causing the cell to further differentiate. The addition of the exogenous differentiating agent, e.g., retinoic acid, enhances PDT activity in the cell.

In another aspect, the invention provides a method of evaluating a subject, e.g., detecting a disorder characterized by unwanted cell proliferation, e.g., cancer. The method is based, in part, on the discovery that the combination of elements of photodynamic therapy (PDT) and differentiation therapy (DT) enhances the fluorescent contrast between neoplastic and normal tissue. This enhancement of fluorescence can improve diagnostic and therapeutic procedures that are based on fluorescence detection. The method includes: providing a differentiating agent to a cell of a subject, e.g., by administration of an exogenous agent; providing the cell with a light emitting agent; activating said agent; and detecting a difference between the cell and a control value, e.g., a difference in the light emitted, e.g., fluorescence, thereby detecting the presence of a disorder characterized by unwanted cell proliferation. The control can be non-disease tissue, e.g., a non-disease tissue in a subject, e.g., adjacent non-disease tissue.

In one embodiment, the light emitting agent is a chemical compound that can be activated to emit light of a first wavelength when activated by light of a second wavelength, e.g., a fluorescent compound. In a preferred embodiment, the fluorescent compound is for example indocyanine green or methylene blue.

In another embodiment, the light emitting agent is a PS. In preferred embodiments: the photosensitizer has a chemical structure that includes multiple conjugated rings that allow for light absorption and photoactivation, e.g., the photosensitizer can produce singlet oxygen upon absorption of electromagnetic irradiation at the proper energy level and wavelength; the photosensitizer can include a porphyrin, porphyrin derivative or analog thereof, e.g., a tetraphyrroles; or the photosensitizer can include chlorin e6, a chlorin derivative or analog thereof. Suitable photosensitizers include photofrin®; synthetic diporphyrins and dichlorins;hydroporphyrins, e.g., chlorins and bacteriochlorins of the tetra(hydroxyphenyl) porphyrin series; phthalocyanines; O-substituted tetraphenyl porphyrins (picket fence porphyrins); 3,1-meso tetrakis (o-propionamido phenyl) porphyrin; Verdins; Purpurins, e.g., tin and zinc derivatives of octaethylpurpurin (NT2), and etiopurpurin (ET2); Chlorins, e.g., chlorin e6, and mono-1-aspartyl derivative of chlorin e6 ; Benzoporphyrin derivatives (BPD), e.g., benzoporphyrin monoacid derivatives, tetracyanoethylene adducts of benzoporphyrin, dimethyl acetylenedicarboxylate adducts of benzoporphyrin, Diels-Adler adducts, and monoacid ring “a” derivative of benzoporphyrin; Low density lipoprotein mediated localization parameters similar to those observed with hematoporphyrin derivative (HPD); sulfonated aluminum phthalocyanine (Pc) sulfonated AlPc disulfonated (AlPcS2) tetrasulfonated derivative sulfonated aluminum naphthalocyanines chloroaluminum sulfonated phthalocyanine (CASP); zinc naphthalocyanines; anthracenediones; anthrapyrazoles; amino anthraquinone; phenoxazine dyes; phenothiazine derivatives; chalcogenapyrylium dyes cationic selena and tellurapyrylium derivatives; ring-substituted cationic PC; pheophorbide a; hematoporphyrin (HP); protoporphyrin; ALA; and ALA-esters, hexyl ester or methyl ester.

In one embodiment, the present method allows for a marked increase in fluorescence upon differentiation of the cell, both for endogenous (autoflourescence) and exogenous fluorescence (induced fluorescence). In one embodiment, auto flourescence and induced fluorescence are measured. In another embodiment, either autoflourescence or induced fluorescence is measured.

In one embodiment, the differentiating agent and light emitting agent, e.g., a fluorescent compound or a PS, are administered individually, or as a combination. In another embodiment, the differentiating agent is administered before administration of the light emitting agent. In another embodiment, the differentiating agent is administered after administration of the light emitting agent.

In a preferred embodiment, the subject has or is at risk for a neoplasia or a dysplasia characterized by unwanted cell proliferation.

In a preferred embodiment, the subject is suspected of having a neoplasia, e.g., a neoplasia of the liver, stomach, prostate, breast, lung, skin, bladder, head and neck, colon, ovary or cervix, and a light emitting agent and a differentiating agent are administered to the subject. In another preferred embodiment the cell is an: epithelial cell; nerve cell; prostate cell; or blood cell, e.g., a white blood cell.

In another embodiment, the method can be used to determine the effectiveness of tumor treatment.

In a preferred embodiment, a PS or the metabolic precursor of a PS is administered to the subject, or is applied to a sample of the subject.

In one embodiment, a compound, e.g., ALA or ALA esters, which causes the accumulation of a PS, the formation of a PS, or is converted to a PS, in the subject's body, or in a sample of the subject, is administered to the subject, or administered to the sample taken from the subject. For example, a compound which causes the accumulation of, the formation of, or which is converted to, a porphyrin, or a porphyrin precursor, is administered to the subject, or to a sample taken from the subject.

In a preferred embodiment, the light emitting agent is coupled to a targeting moiety. Examples of targeting moieties include antibodies, or antigen binding fragments thereof, and ligands for cell surface molecules.

The differentiating agent can be, e.g., chemotherapeutic agent, a hormonal agent, e.g., a retinoid or vitamin D, or an agent which increases levels of retinoic acid, e.g., liarozole or Liazal retinoic acid; or an antidiabetic compound, e.g., troglituzone, or a ligand for a transcription factor, e.g., transcription factor PPAR gamma.

In one embodiment, the differentiating agent is administered before administration of light emitting agent, e.g., the differentiating agent is administered so as to allow the cell of interest to differentiate, e.g., the differentiating agent is administered to the subject, e.g., 4 weeks, 2 weeks, 1 week, 72 hours, 48 hours, or 24 hours, or less, before the PS is administered. In another embodiment, the differentiating agent is administered consecutively with the light-emitting agent.

In a preferred embodiment, a cell suspected of being neoplastic is induced to differentiate and the light-emitting agent is supplied such that it is present while the cells are in a state of induced differentiation. The activating light can also be supplied while the cell is in the induced state.

In a preferred embodiment, the method includes detecting the stage or type of cell tumor, detecting the extent or distribution of cells characterized by unwanted proliferation, e.g., detecting the margin of a tumor, or detecting the boundary between diseased and nondisease tissue.

In a preferred embodiment, the method further includes treatment of a tissue identified as having unwanted cell proliferation, e.g., surgical removal of the tissue.

In a preferred embodiment, the method includes making a first determination or evaluation of the distribution or extent of unwanted cell proliferation in a tissue, and administering a treatment, e.g., surgical removal, laser ablation etc, and making a second determination or evaluation of the distribution or extent of unwanted cell proliferation in a tissue, and administering further treatment, if necessary. This method can be repeated until the tissue is free of cells that are characterized by unwanted cell proliferation.

In a preferred embodiment, the method includes detecting unwanted proliferation of cells in a first tissue or structure, e.g., liver, stomach, prostate, breast, skin, bladder, head and neck, colon, ovary or cervix, and determining if the unwanted cell proliferation has spread to a second tissue or structure.

Another aspect of the present invention features a method of treating a cell of a subject having a disorder characterized by unwanted cell proliferation, such as cancer. The method comprises the steps of inducing differentiation in a cell with a chemotherapeutic agent, providing the cell with a photosensitizer, activating the photosensitizer, thereby inhibiting or treating unwanted cell proliferation. Administration of the chemotherapeutic agent and the photosensitizer can be sequential, in either order, or concomitant.

In a preferred embodiment, the chemotherapeutic agent is selected from the group consisting of cyclophosphamide, ifosfamide, thiotepa, busulfan, carmustine, dabarcazine, methotrexate, 5-fluorouracil, 5-fluorodeoxyuridine, cytosine arabinoside (AraC), mercaptopurine, thioguanine, fludarabine phosphate, azathioprine, vinblastine, vincristine, vindesine, vinorelbine, paclitaxel, etoposide, teniposide, actinomycin D, daunorubicin, doxorubicin, idarubicin, bleomycin, mithramycin, mitomycin, cisplatin, carboplatin, and hydroxyurea.

Administration of the light-emitting agent, photosensitizer or differentiating agent can be local or systemic. The administration can be by any suitable route, including topical, intravenous, intraarticular, subcutaneous, intramuscular, intraventricular, intracapsular, intraspinal, intraperitoneal, topical, intranasal, oral, buccal, rectal or vaginal. The preferred route of administration will depend on the location of the cells suspected of having unwanted cell proliferation.

Parenteral formulations may be in the form of liquid solutions or suspensions; for oral administration, formulations may be in the form of tablets or capsules; and for intranasal formulations, in the form of powders, nasal drops, or aerosols.

As used herein, a “photosensitizer” refers to a substance, which upon irradiation with electromagnetic energy of the appropriate wavelength, usually light of the appropriate wavelength, produces a cytotoxic effect and/or produce a wavelength, which can be detected.

The term “subject,” as used herein, refers to a living animal or human having a cell that is a target for photodynamic therapy. The subject can be a mammal, including humans and non-human mammals such as dogs, cats, pigs, cows, sheep, goats, horses, rats, and mice. The subject may formerly have been treated by chemotherapy.

As used herein, a “light-emitting agent” refers to an agent that upon irradiation emits a photon, e.g., a luminescent or fluorescent agent. Preferred light emitting agents are fluorescent compounds and PS's.

As used herein, the term “neoplastic” refers to cells having the capacity for autonomous growth, i.e., an abnormal state or condition characterized by rapidly proliferating cell growth.

As used herein, a “disorder characterized by unwanted cell proliferation” refers to both benign and malignant disoders. Examples of disorders characterized by unwanted cell proliferation include cancerous growths or oncogenic processes, metastatic tissues or malignantly transformed cells, tissues, or organs, and non-pathologic hyperproliferative cells, e.g., proliferation of cells associated with wound repair. Additional examples include hematopoietic neoplastic disorders and includes diseases involving hyperplastic/neoplastic cells of hematopoietic origin, e.g., arising from myeloid, lymphoid or erythroid lineages, or precursor cells thereof.

Unless otherwise defined, all technical and scientific terms used herein have the same meaning as commonly understood by one of ordinary skill in the art to which this invention belongs. Although methods and materials similar or equivalent to those herein can be used in the practice or testing of the present invention, the preferred methods and materials are described below. All publications, patent applications, patents, and other references mentioned herein are incorporated by reference. In addition, the materials, methods, and examples are illustrative only and not intended to be limiting.

### BRIEF DESCRIPTION OF THE DRAWINGS

FIG. 1 is a graph of ALA induced PpIX production (as % of control) in R1881 treated LNCaP cells.

FIG. 2 is a graph of % survival in LNCaP±10⁻⁷ M R1881 (3 d), 10 J/cm² 514 nm.

FIG. 3 is a graph of % survival in LNCaP±10⁻⁷ M R1881 (3 d) 0.3 mM ALA (4 h).

FIGS. 4A-B are photographs depicting control LNCaP cells treated with ALA (FIG. 4A) and LNCaP cells that were pretreated with R1881 and also treated with ALA in vitro (FIG. 4B).

FIG. 5A-B are photographs depicting tumor sections consisting of control LNCaP cells from mice treated with ALA (FIG. 5A) and a tumor section from mice pretreated with R 1881 and also treated with ALA in vivo (FIG. 5B).

FIG. 6A is a graph depicting light-induced cytotoxicity of LNCaP cells as measured by MTT conversion assay.

FIG. 6B depicts a colony formation assay of LNCaP cells after enhancement of PDT by chemotherapy.

### DETAILED DESCRIPTION OF THE INVENTION

Photodynamic therapy is an approved modality for the treatment of certain cancers. It usually includes the localization of a photoactivatable chemical in tumor in excess of surrounding tissues. There are many ways of achieving this, many of which depend on the packaging of the photosensitizing agent with a targeting moiety.

The integration of PDT and DT enhances the effect of treatment of unwanted cell proliferation. While not wising to be bound by theory, it is believed that the administration of a differentiating agent to a cell makes the cell more susceptible to PDT.

Methods of the invention will extend the benefits of differentiation therapy, by applying it together with PDT. Methods of the invention differ, inter alia, from conventional differentiation therapy in the sense that in a method of the invention alter the state of the tumor cells need only be temporary, i.e., the induced state need only last long enough to make them more susceptible to PDT.

Methods of the invention allow treatment for a variety of conditions that are characterized by unwanted cell proliferation, e.g., cancer related pathologies or psoriasis that will lead to a significantly more efficient eradication of cells by first altering the state of the cells so as to make them more susceptible to PDT and to take more of the photosensitizer. Cancer-related pathologies in the context of the present invention include, but are not limited to, tumors and pathologies involving tumorigenesis, leukemias, lymphomas, melanomas, sarcomas, virus-related cancers, and any other known cancers. Moreover, the integration of PDT and DT also enhances the fluorescent contrast between neoplastic and normal tissue and therefore can be used to evaluate a disorder characterized by unwanted cell proliferation. This discovery provides an enhanced method for analyzing, e.g., diagnosing, a subject suspected of having a disorder characterized by unwanted cell proliferation. The diagnostic methods provide a means of distinguishing normal from disease state, e.g., transformed tissue. In a preferred embodiment, the method allows the detection of early cancers and neoplasias, e.g., neoplasias of the liver, stomach, prostate, breast, lung, skin, nerve cells, bladder, head and neck, colon, ovary or cervix, and dysplasias.

Photosensitizers

A photosensitizer is a substance that, upon irradiation with electromagnetic energy of the appropriate wavelength, usually light of the appropriate wavelength, produces a cytotoxic effect. Many photosensitizers produce singlet oxygen. Upon electromagnetic irradiation at the proper energy level and wavelength, such a photosensitizer molecule is converted to an energized form. The energized form can react with atmospheric 02, such that upon decay of the photosensitizer to the unenergized state, singlet oxygen is produced. Singlet oxygen is highly reactive, and is toxic to a proximal target organism.

The life-time of its triplet energized state should be of sufficient duration (e.g., several microseconds) to permit interaction with neighboring molecules to produce cytotoxic species. A photosensitizer composition should efficiently absorb electromagnetic energy of the appropriate wavelength with high quantum yield to efficiently generate the energized form of the photosensitizer. Toxicity to the target cells should increase substantially, preferably 10-fold, 100-fold, or even more preferably 1,000-fold, upon irradiation. A photosensitizer should exhibit low background toxicity, i.e., low toxicity in the absence of irradiation with energy of the appropriate wavelength.

Other preferred properties of a photosensitizer include high solubility and stability in appropriate solvents. For example, a photosensitizer should be soluble under conditions used to couple it to the targeting moiety or backbone. Desired solubility properties will differ with the conditions chosen for the reaction but solubility in DMSO, water, ethanol, or a mixture of water and DMSO or in ethanol, such as DMSO: H20, or in ethanol:water 5%, 10% or 15% can be useful. Solubility is preferably 50 pg/ml, 100 pg/ml, 500 pg/ml, 1 mg/ml or 10 mg/ml in an aqueous solvent or ethanol:water solvent.

When conjugated to a targeting moiety, the resulting photosensitizer:targeting moiety conjugate should be soluble under physiological conditions, in aqueous solvents containing appropriate carriers and excipients, or other delivery systems such as in liposomes. The molecules of the invention may be delivered as free photosensitizer:targeting moiety compositions in solution, and may be delivered also in various formulations including, but not limited to, liposome, peptide/polymer-bound, or detergent-containing formulations.

The compositions of the invention should be stable during the course of at least a single round of treatment by continuous or pulsed irradiation, during which each molecule of the composition would preferably be repeatedly excited to the energized state, undergoing multiple rounds of generation of singlet oxygen. Preferable stability of a photosensitizer conjugate molecule is survival of 10%, 50%, 90%, 95%, or 99% of molecules in active form for 1 hour, for 30 min, 15 min or for at least 1 min at 37° C., under physiological conditions.

Photosensitizers include, but are not limited to, hematoporphyrins, such as hematoporphyrin HCl and hematoporphyrin esters (Dobson, J. and M. Wilson, Archs. Oral Biol. 37:883-887); dihematophorphyrin ester (Wilson, M. et al., 1993, Oral Microbiol. Immunol. 8:182-187); hematoporphyrin IX (Russell et al., 1991, Can J. App. Spectros. 36:103-107, available from Porphyrin Products, Logan, Utah) and its derivatives; 3,1-meso tetrakis (o-propionamidophenyl)porphryrin; hydroporphyrins such as chlorinand bacteriochlorin of the tetra(hydroxyphenyl)porphyrin series, and synthetic diporphyrins and dichlorins; o-substituted tetraphenyl porphyrins (picket fence porphyrins); chlorin e6 monoethylendiamine monamide (CMA Goff, B. A. et al., 1994, 70:474-480, available from Porphyrin Products, Logan, Utah); mono-1-aspartyl derivative of chlorin e6, and mono- and di-aspartyl derivatives of chlorin e6; the hematoporphyrin mixture Photofrin II (Quadra Logic Technologies, Inc., Vancouver, BC, Canada); benzophorphyrin derivatives (BPD), including benzoporphyrin monoacid Ring A (BPD-MA), tetracyanoethylene adducts, dimethyl acetylene dicarboxylate adducts, Diels-Adler adducts, and monoacid ring “A” derivatives; a naphthalocyanine (Biolo, R., 1994, Photochem. and Photobiol. 5959:362-365); a Zn(II)-phthalocyanine (Shopora, M. et al., 1995, Lasers in Medical Science 10:43-46); toluidine blue O (Wilson, M. et al., 1993, Lasers in Medical Sci. 8:69-73); aluminum sulfonated and disulfonated phthalocyanine ibid.; and phthalocyanines without metal substituents, and with varying other substituents; a tetrasulfated derivative; sulfonated aluminum naphthalocyanines; methylene blue (ibid.); nile blue; crystal violet; azure (3 chloride; and rose bengal (Wilson, M., 1994, Intl. Dent. J. 44:187-189). Numerous photosensitizer entities are disclosed in Wilson, M. et al., 1992, Curr. Micro. 25:77-81, and in Okamoto, H. et al., 1992, Lasers in Surg. Med. 12:450-485.

Other potential photosensitizer compositions include but are not limited to, pheophorbides such as pyropheophorbide compounds, anthracenediones; anthrapyrazoles; aminoanthraquinone; phenoxazine dyes; phenothiazine derivatives; chalcogenapyrylium dyes including cationic selena- and tellura-pyrylium derivatives; verdins; purpurins including tin and zinc derivatives of octaethylpurpurin and etiopurpurin; benzonaphthoporphyrazines; cationic imminium salts; and tetracyclines.

The suitability of a photosensitizer for use in a conjugate can be determined by methods described herein or by methods known to those skilled in the art.

The efficiency with which a photosensitizer oxidizes a target molecule is a measure of the usefulness. The efficiency of the oxidation of a target molecule by a photosensitizer can be determined in vitro. Examples of substrates include 4-nitroso-N,N-dimethylaniline (RNO; Hasan, T. et al., 1987, Proc. AACR 28:395 Abstr. 1,568), and tryptophan or histidine (Lambert, C. R. et al., 1986, Photochem. Photobiol. 44:595-601). In these assays, ability of a candidate photosensitizer to “bleach” the substrate can be monitored spectroscopically. The advantage of a chemical assay is that a large number of putative photosensitizer compositions can be simultaneously screened. Parameters that can be varied include photosensitizer concentration, substrate concentration, optimal intensity of irradiation, and optimal wavelength of irradiation. High through-put technologies including plastic multiwell dishes, automated multi-pipetters, and on-line spectrophotometric plate readers can be used. Undesirable candidates, e.g., compositions having high backgrounds under unirradiated conditions, inefficient energy capture or reactive potential, can be identified and eliminated.

A PS can also be provided by administering a compound to the subject wherein the compound causes the production, e.g., the accumulation, of a PS. For example, administering a compound which results in an inverse in a PS, e.g., a porphyrin or precursor thereof. A preferred example is ALA-induced proto porphyrin IX accumulation. See, e.g., Ortel et al., 1998, Journal of Cancer 77:1744, Loreby incorporated by reference. The PS can be administered by any means known in the art, e.g., administration can be systemic, local, parenteral, e.g., intravenous, intradermal, subcutaneous, oral (e.g., inhalation), transdermal (topical), transmucosal, and rectal administration.

Irradiation

Irradiation of the appropriate wavelength for a given compound may be administered by a variety of methods. These methods include but are not limited to the administration of laser, nonlaser, or broadband light. Irradiation can be produced by extracorporeal or intraarticular generation of light of the appropriate wavelength. Light used in the invention may be administered using any device capable of delivering the requisite power of light including, but not limited to, fiber optic instruments, arthroscopic instruments, or instruments which provide transillumination. Delivery of light to a body cavity can be accomplished with flexible fiber optics.

Targeting Moieties

Desirable characteristics for the targeting moieties include: specificity for one or more cell or tissue types, affinity and avidity for such targets, and stability with respect to conditions of coupling reactions and the physiology of the organ or tissue of use.

The targeting moiety, when incorporated into a conjugate molecule of the invention, should be nontoxic to the cells of the subject.

Targeting moieties can be selected from the sequences of naturally occurring proteins and peptides, from variants of these peptides, and from biologically or chemically synthesized peptides. Naturally occurring peptides which have affinity for a target cell can provide sequences from which additional peptides with desired properties, e.g., increased affinity or specificity, can be synthesized individually or as members of a library of related peptides. Such peptides can be selected on the basis of affinity for the target cell or tissue.

Naturally occurring peptides with affinity for a target cell useful in methods and compounds of the invention, include ligands for cell surface molecules, e.g., receptors. Molecules having a specific affinity for a target cells are preferred. Such molecules can include antibodies (or antigen binding fragments thereof) and ligands for cell surface molecules. E.g., the art is replete with monoclonal antibodies specific for prostate or cancerous prostate tissue. Ligands which bind hormone or growth factor receptors can be used in methods described herein.

Coupling Technologies

The light emitting agent, e.g., a PS, can be coupled to a targeting moiety in order to target the PS to a particular cell type. Often it is desirable to couple the light emitting agent, e.g., a PS, and the targeting molecule via a back-bone linker. Different methods are described below which can be used to couple a light emitting agent, e.g., PS, to a targeting moiety.

The term “coupling agent” as used herein, refers to a reagent capable of coupling a light emitting agent, e.g., a photosensitizer to a targeting moiety, or light emitting agent, e.g., a photosensitizer, or a targeting moiety to a “backbone” or “bridge” moiety. Any bond which is capable of linking the components such that they are stable under physiological conditions for the time needed for administration and treatment is suitable, but covalent linkages are preferred. The link between two components may be direct, e.g., where a light emitting agent, e.g., a photosensitizer, is linked directly to a targeting moiety, or indirect, e.g., where a light emitting agent, e.g., a photosensitizer, is linked to an intermediate, e.g., linked to a backbone, and that intermediate being linked to the targeting moiety. A coupling agent should function under conditions of temperature, pH, salt, solvent system, and other reactants that substantially retain the chemical stability of the photosensitizer, the backbone (if present), and the targeting moiety.

A coupling agent can link components without the addition to the linked components of elements of the coupling agent. Other coupling agents result in the addition of elements of the coupling agent to the linked components. For example, coupling agents can be crosslinking agents that are homo- or hetero-bifunctional, and wherein one or more atomic components of the agent can be retained in the composition. A coupling agent that is not a cross-linking agent can be removed entirely during the coupling reaction, so that the molecular product can be composed entirely of the photosensitizer, the targeting moiety, and a backbone moiety (if present). Many coupling agents react with an amine and a carboxylate, to form an amide, or an alcohol and a carboxylate to form an ester. Coupling agents are known in the art, see, e.g., M. Bodansky, “Principles of Peptide Synthesis”, 2nd ed., referenced herein, and T. Greene and P. Wuts, “Protective Groups in Organic Synthesis,” 2nd Ed, 1991, John Wiley, N.Y. Coupling agents should link component moieties stably, but such that there is only minimal or no denaturation or deactivation of the light emitting agent, e.g., a photosensitizer, or the targeting moiety.

The photosensitizer conjugates of the invention can be prepared by coupling the light emitting agent, e.g., a photosensitizer, to targeting moieties using methods described in the following examples, or by methods known in the art. A variety of coupling agents, including cross-linking agents, can be used for covalent conjugation. Examples of cross-linking agents include N,N′-dicyclohexylcarbodiimide (DCC; Pierce), N-succinimidyl-S-acetyl-thioacetate (SATA), N-succinimidyl-3-(2-pyridyldithio)propionate (SPDP), orthophenylenedimaleimide (o-PDM), and sulfosuccinimidyl 4-(N-maleimidomethyl) cyclohexane-1-carboxylate (sulfo-SMCC). See, e.g., Karpovsky et al. .1. Exp. Med. 160:1686, 1984; and Liu, M A et al., Proc. Nad. Acad. Sci. USA 82:8648, 1985. Other methods include those described by Paulus, Behring Ins. Mitt., No. 78, 118-132, 1985; Brennan et al. Science 229:81-83, 1985, and Glennie et al., J. Immunol., 139: 2367-2375, 1987. A large number of coupling agents for peptides and proteins, along with buffers, solvents, and methods of use, are described in the Pierce Chemical Co. catalog, pages T-155-T-200, 1994 (3747 N. Meridian Rd., Rockford Ill., 61105, U.S.A.; Pierce Europe B.V., P.O. Box 1512, 3260 BA Oud Beijerland, The Netherlands), which catalog is hereby incorporated by reference.

DCC is a useful coupling agent (Pierce #20320; Rockland, Ill.). It promotes coupling of the alcohol NHS to chlorin e6 in DMSO (Pierce #20684), forming an activated ester which can be cross-linked to polylysine. DCC (N,N′-dicyclohexylcarbodiimide) is a carboxy-reactive cross-linker commonly used as a coupling agent in peptide synthesis, and has a molecular weight of 206.32. Another useful cross-linking agent is SPDP (Pierce #21557), a heterobifunctional cross-linker for use with primary amines and sulfhydryl groups. SPDP has a molecular weight of 312.4, a spacer arm length of 6.8 angstroms, is reactive to NHS-esters and pyridyldithio groups, and produces cleavable cross-linking such that, upon further reaction, the agent is eliminated so the light emitting agent, e.g., a photosensitizer, can be linked directly to a backbone or targeting moiety. Other useful conjugating agents are SATA (Pierce #26102) for introduction of blocked SH groups for two-step cross-linking, which is deblocked with hydroxylamine-HCl (Pierce #26103), and sulfo-SMCC (Pierce #22322), reactive towards amines and sulfhydryls. Other cross-linking and coupling agents are also available from Pierce Chemical Co. (Rockford, Ill.). Additional compounds and processes, particularly those involving a Schiff base as an intermediate, for conjugation of proteins to other proteins or to other compositions, for example to reporter groups or to chelators for metal ion labeling of a protein, are disclosed in EPO 243,929 A2 (published Nov. 4, 1987).

Light emitting agents, e.g., photosensitizers, which contain carboxyl groups can be joined to lysine E-amino groups in the target polypeptides either by preformed reactive esters (such as N-hydroxy succinimide ester) or esters conjugated in situ by a carbodiimide-mediated reaction. The same applies to photosensitizers that contain sulfonic acid groups, which can be transformed to sulfonyl chlorides which react with amino groups. Photosensitizers which have carboxyl groups can be joined to amino groups on the polypeptide by an in situ carbodiimide method. Light emitting agents, e.g., photosensitizers, can also be attached to hydroxyl groups, of serine or threonine residues or to sulfhydryl groups of cysteine residues.

Methods of joining components of a conjugate, e.g., coupling polyamino acid chains bearing photosensitizers to antibacterial polypeptides, can use heterobifunctional cross linking reagents. These agents bind a functional group in one chain and to a different functional group in the second chain. These functional groups typically are amino, carboxyl, sulfliydryl, and aldehyde. There are many permutations of appropriate moieties which will react with these groups and with differently formulated structures, to conjugate them together. See the Pierce Catalog, and Merrifield, R. B., et al. Ciba Found Symp. 186:5-20, 1994.

The production and purification of light emitting agent:targeting moiety conjugates can be practiced by methods known in the art. Yield from coupling reactions can be assessed by spectroscopy of product eluting from a chromatographic fractionation in the final step of purification. The presence of uncoupled photosensitizer and reaction products containing the photosensitizer can be followed by the physical property that the photosensitizer moiety absorbs light at a characteristic wavelength and extinction coefficient, so incorporation into products can be monitored by absorbance at that wavelength or a similar wavelength. Coupling of one or more photosensitizer molecules to a targeting moiety or to a backbone shifts the peak of absorbance in the elution profile in fractions eluted using sizing gel chromatography, e.g., with the appropriate choice of Sephadex G50, 6100, or 6200 or other such matrices (Pharmacia-Biotech, Piscataway N.J.). Choice of appropriate sizing gel, for example Sephadex gel, can be determined by that gel in which the photosensitizer elutes in a fraction beyond the excluded volume of material too large to interact with the bead, i.e., the uncoupled starting photosensitizer composition interacts to some extent with the fractionation bead and is concomitantly retarded to some extent. The correct useful gel can be predicted be predicted from the molecular weight of the uncoupled photosensitizer. The successful reaction products of photosensitizer compositions coupled to additional moieties generally have characteristic higher molecular weights, causing them to interact with the chromatographic bead to a lesser extent, and thus appear in fractions eluting earlier than fractions containing the uncoupled photosensitizer substrate. Unreacted photosensitizer substrate generally appears in fractions characteristic of the starting material, and the yield from each reaction can thus be assessed both from size of the peak of larger molecular weight material, and the decrease in the peak of characteristic starting material. The area under the peak of the product fractions is converted to the size of the yield using the molar extinction coefficient.

The product can be analyzed using NMR, integrating areas of appropriate product peaks, to determine relative yields with different coupling agents. A red shift in absorption of a photosensitizer of several nm has often been observed following coupling to a polyamino acid. Coupling to a larger moiety such as a protein might produces a comparable shift, as coupling to an antibody resulted in a shift of about 3-5 nm in that direction compared to absorption of the free photosensitizer. Relevant absorption maxima and extinction coefficients in 0.1 M NaOH/1% SDS are, for chlorin e6, 400 run and 150,000 M-1, cm-1, and for benzoporphyrin derivative, 430 nm and 61,000 M-1, cm-1.

Differentiation Therapy

Neoplasia involves the loss of normal regulatory mechanisms often associated with the differentiated state. Among a host of strategies to deal with neoplasia, differentiation therapy takes advantage of the fact that normal regulation can sometimes be restored by inducing terminal differentiation of the cancer cells (Carducci et al., Seminars in Oncology. 23: 56-62, 1996). The best-known example of differentiation therapy is probably the use of retinoids in acute promyelocytic leukemia. For that particular malignancy, administration of retinoic acid induces immature promyelocytic cells to differentiatiate along the pathway toward mature neutrophils, producing cells that are less proliferative and more responsive to adjunctive chemotherapy (Degos et al., Blood. 85: 2643-53, 1995). The newest example of differentiation therapy may be the use of antidiabetic drugs (troglitazone), ligands for the nuclear transcription factor PPARgamma, to stimulate terminal differentiation of malignant breast epithelial cells (Elstneret al., Proc Natl Acad Sci USA. 95: 8806-11, 1998 and Mueller et al., Mol Cell 1: 465-70, 1998). For prostate carcinoma, the notion of differentiation therapy has stemmed from observations that hormonal agents, principally retinoids and vitamin D, can induce differentiation markers in cell lines derived from prostate tumors, e.g. the LNCaP line. Clinically, an agent (liarozole, Liazal) which promotes cell differentiation by increasing intratumoral levels of retinoic acid is now in early clinical trials, and appears to have some promise for the treatment of prostate cancer. Examples of other differentiating agents include but are not limited to, polar/apolar compounds such as hexamethylene bisacetamide; vitamin D analogs including 1,25-(OH).sub.2 D.sub.3; histone hyperacetylators such as sodium butyrate and prodrugs thereof, sodium propionate and trichostatin A; hormones such as glucocorticoids; antioxidants such as PDTC; peroxisome proliferators such as clofibrate; and miscellaneous differentiating agents such as phenylacetate and phenylbutyrate.

In a preferred embodiment, the differentiating agent is not a cytokine.

For more information on DT, see Pierce et al. 1988, Cancer Res.:48:1996, and Blutt et al. 1997. Endocrinology 138:1491, both of which are incorporated by reference.

Fluorescence Photodetection, Photodynamic Therapy and Differentiation Therapy

Fluorescence photodetection of cancers involves the excitation of a chromophore with light and monitoring the emitted light as fluorescence. The combination of treating a cell in a subject suspected of having a neoplasia with a differentiating agent, and a light emitting agent, e.g., a photosensitizer or a fluorescent compound, allows for a marked increase in light emission upon differentiation of the cell, e.g., both for endogenous (autoflourescence) and exogenous fluorescence (induced fluorescence). This method can be preformed in vivo, or a sample can be taken from a subject and performed ex vivo.

The enhancement of contrast afforded by this method is useful for the in vivo detection of early neoplasias. Moreover, the improved contrast also provides a method of enhancing the delineation of margins of resection. For example, the present method can be used as a guide to a surgeon to identify the precise location of tumor growth, thereby enabling the surgeon to precisely treat, e.g., remove the tumor. In another example, the method can be used to determine the effectiveness of tumor treatment. For example, following tumor treatment, the efficiacy of the treatment can be determined by using the method to determine any residual or new tumor growth, and if necessary the tumor can be retreated.

The method can be used to detect and/or treat any cell characterized as having unwanted cell proliferation. Examples of such cells include all types of cancerous growths or oncogenic processes, metastatic tissues or malignantly transformed cells, tissues, or organs, and non-pathologic hyperproliferative cells, e.g., proliferation of cells associated with wound repair. Additional examples of proliferative disorders include hematopoietic neoplastic disorders and includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML).

Standard methods of detecting and quantitating light emission, e.g., fluorescence, in vivo are known, e.g., endoscopic fluorescence microscopy or laser-induced flouresence (LIF) spectrometer, can be used in the method of the invention. For further details on these standard methods see e.g., U.S. patent Ser. No. 5,769,792; or Bedwell et al., Br. J. Cancer, 65:818-824, 1992.

Chemotherapeutic Agents

Chemotherapeutic agents can be used as differentiation agents. Sequential or concomitant treatment with differentiation inducing agents such as hormones, vitamin A, vitamin D and chemotherapeutic agents leads to an increased sensitivity of cells to PDT. Because differentiation is usually associated with growth arrest, treatment with chemotherapy can have a similar effect on cellular sensitivity to PDT.

In one embodiment, the invention relates to a method of treating a subject having a disorder characterized by unwanted cell proliferation, the method comprising the steps of administering a chemotherapeutic agent to a subject in an amount sufficient to induce differentiation in the cell; administering a photosensitizer; activating the photosensitizer; inhibiting unwanted cell proliferation. “Inhibiting” unwanted cell proliferation is to reduce or eliminate any undesired cell proliferation such that there is a beneficial health effect in the subject.

A variety of known chemotherapeutic agents can be used to sensitize cancer cells to PDT. Representative examples of chemotherapeutic agents are described below, but are not limited to these agents. Alkylating agents disturb the fundamental mechanisms regulating cell proliferation, particularly DNA synthesis and cell division. The capacity of these drugs to interfere with DNA integrity and function in rapidly proliferating tissues provides the basis for their therapeutic applications and for many of their toxic properties. Such examples of alkylating agents that have known metabolites include cyclophosphamide, ifosfamide, thiotepa (triethylene thiophosphoramide), busulfan, carmustine, and dabarcazine, but are not limited to these aforementioned agents.

Cyclophosphamide is first converted to 4-hydroxycyclophosphamide, which is in a steady state with the acyclic tautomer, aldophosphamide. In tumor cells, the aldophosphamide may cleave spontaneously, generating stoichiometric amounts of phosphamide mustard and acrolein. The former is primarily responsible for anti-tumor effects.

Thiotepa and its primary metabolite, triethylenephosphoramide (TEPA), are capable of forming DNA cross-links. Busulfan is an alkyl sulfonate and is metabolized to methanesulfonic acid. Carmustine is capable of inhibiting the synthesis of DNA, RNA, and protein. It kills cells in all phases of the cell cycle. Carmustine is metabolized to form alkylated derivatives. Dacarbazine functions as a methylating agent after metabolic activation in the liver, and also kills cells in all phases of the cell cycle. Dacarbazine resistance has been ascribed to the repair of methylated guanine bases in DNA by guanine O⁶-alkyl transferase.

Antimetabolites interfere with the normal physiological processes that ultimately impinge on DNA synthesis, including inhibition of de novo purine and pyrimidine biosynthesis, as well as folic acid production. Antimetabolites encompass, but are not limited to, the following: folic acid analogs such as methotrexate; pyrimidine analogs such as 5-fluorouracil, 5-fluorodeoxyuridine, cytosine arabinoside, mercaptopurine, thioguanine, and fludarabine phosphate.

Methotrexate is an inhibitor of dihydrofolate reducatse, and also directly inhibits the folate-dependent enzymes of de novo purine and thymidylate synthesis. Fluorouracil (5-FU) and fluorodeoxyuridine inhibit the biosynthesis of pyrimidine nucleotides and interfere with synthesis of nucleic acids. Fluorodeoxyuridine is converted by thymidine or deoxyuridine phosphorylases into 5-FU. Cytarabine, or AraC, is used to treat acute myelocytic leukemia, and is an analog of 2′-deoxycytidine. AraC is degraded as the inactive, deaminated product, arabinosyl uracil. Mercaptopurine, azathioprine, as well as thioguanine, are purine analogs that interfere with nucleotide biosynthesis. Fludarabine phosphate is a fluorinated deamination-resistant nucleotide analog of the antiviral agent vidarabine.

Natural products include anti-mitotic drugs, which block cells from undergoing mitosis. The biological activities of these drugs can be explained by their ability to bind specifically to tubulin and to block the ability of the protein to polymerize into microtubules. Anti-mitotic drugs include but are not limited to the vinca alkaloids vinblastine, vincristine, vindesine, and vinorelbine. Paclitaxel and its analog doetaxel are exceptions in that they promote rather than inhibit microtubule formation. Paclitaxel instead binds specifically to the β-subunit of tubulin and appears to antagonize the disassembly of this key cytoskeletal protein, which is then followed by mitotic arrest.

Epipodophyllotoxins do not arrest cells in mitosis; rather these compounds form a ternary complex with topoisomerase II and DNA. This complex results in double-stranded DNA breaks, but the strand passage and resealing of the break that normally follow topoisomerase binding to DNA are inhibited by the drug. The enzyme remains bound to the free end of the broken DNA strand, leading to an accumulation of DNA breaks and cell death. Epipodophyllotoxins include etoposide, and teniposide.

Antibiotics such as actinomycin D, daunorubucin, doxorubicin, and idarubicin intercalate with double-stranded DNA and therefore block transcription. Bleomycin, on the other hand, causes fragmentation of DNA, resulting in chromosomal aberrations, chromatid breaks, gaps, fragments, and translocations. Bleomycin cleaves DNA by interaction with oxygen and Fe2+. Mithramycin (plicamycin) and mitomycin are other antibiotics that promote cross-linking and breakage of the DNA strands.

Platinum-containing agents such as cisplatin and carboplatin can be used to promote the formation of DNA adducts and cross-linking of guanine and adenine. The DNA adducts formed by platinum containing compounds inhibit DNA replication and transcription, leading to breakage and miscoding.

Hydroxyurea is representative of a group of compounds that have as their primary site of action the enzyme ribonucleotide diphosphate reductase. This enzyme catalyzes the reductive conversion of ribonucleotides to deoxyribonucleotides. Procarbazine is a DNA methylating agent that must undergo metabolic activation to generate the proximal cytotoxic reactants. These reactants are responsible for DNA methylation.

Administration of the light-emitting agent, photosensitizer or differentiating agent, including a chemotherapeutic agent, can be local or systemic. The administration can be by any suitable route, including topical, intravenous, intraarticular, subcutaneous, intramuscular, intraventricular, intracapsular, intraspinal, intraperitoneal, topical, intranasal, oral, buccal, rectal or vaginal. The preferred route of administration will depend on the location of the cells suspected of having unwanted cell proliferation. Selection of dosages and schedules of administration are well within the ability of one skilled in the art.

In one embodiment, the differentiating agent is administered before administration of light emitting agent, e.g., the differentiating agent is administered so as to allow the cell of interest to differentiate, e.g., the differentiating agent is administered to the subject, e.g., 4 weeks, 2 weeks, 1 week, 72 hours, 48 hours, or 24 hours, or less, before the PS is administered. In another embodiment, the differentiating agent is administered sequentially with the light-emitting agent. Administration of a differentiating agent, including a chemotherapeutic agent, and the photosensitizer can be sequential, in either order, or concomitant.

### EXAMPLES

### Example 1

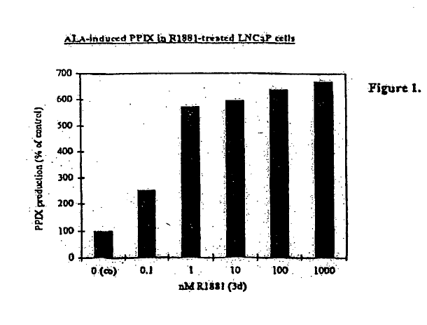

ALA-induced PpIX formation by LNCaP human prostate cancer cells. The cells were incubated for four hours with 0.3 mM ALA after 72 hours of pretreatment with the synthetic androgen R1881 at concentrations between 10⁻¹⁰ to 10⁻⁶. The PpIX formation is recorded as % of cells without androgen treatment and shows a significant increase in R1881-treated cells. See FIG. 1.

### Example 2

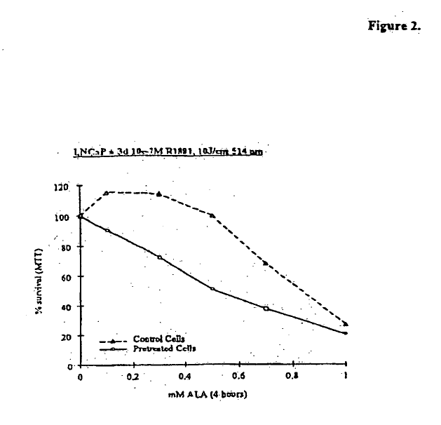

ALA-induced PpIX for PDT of LNCaP cells with (circles) or without (triangles) pretreatment for 72 hours with 10⁻⁷ M R1881. The cells were incubated with ALA concentrations from 0.1 to 1.0 mM for four hours prior to exposure to 10 J/cm² of 514 nm light. Reduced survival of R1881-pretreated cells. See FIG. 2.

### Example 3

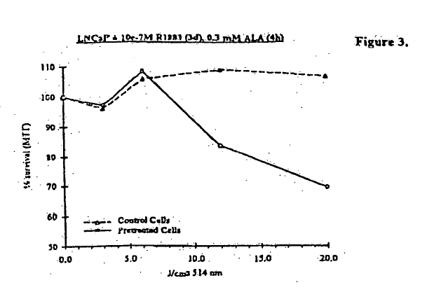

PDT using ALA-induced PpIX in LNCaP cells with (circles or without (triangles) pretreatment for 72 hours with 10-7M 81881. The cells were incubated with 0.3 Mm ALA for four hours prior to exposure to 3-20 J/cm2 of 514 nm light. Reduced survival of R1881 pretreated cells was seen. See FIG. 3.

PDT using ALA-induced PpIX in LNCaP cells with (circles) or without (triangles) pretreatment for 72 hours with 10⁻⁷ M R1881. The cells were incubated with 0.3 mM ALA for four hours prior to exposure to 3-20 J/cm² of 514 nm light. Reduced survival of R1881 pretreated cells was seen. See FIG. 3.

### Example 4

Fluorescence micrograph of ALA-induced PpIX in human skin which was incubated in ALA for 4 hours, showed an increase of fluorescence signal from bottom to top epidermal layers.

### Example 5

This example shows increased PPIX formation in LNCaP cells after R1881 treatment in vitro.

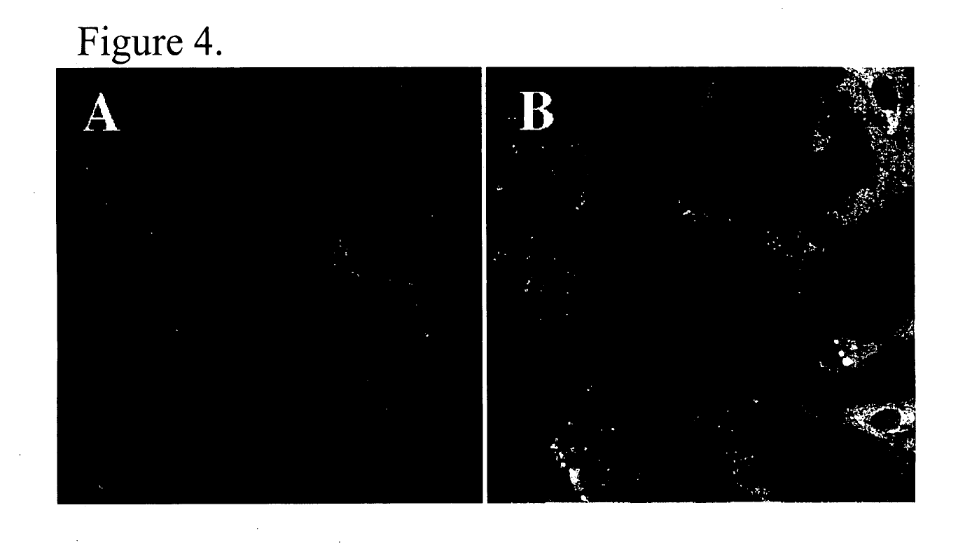

LNCaP cells were pretreated with 10⁻⁷ M R1881 for 72 hours or with vehicle, and then incubated with 0.3 mM ALA. At 4 hours the living cells were imaged using the confocal laser scanning microscope. Excitation was at 488 nm and both green (autofluorescence) and red (PPIX) fluorescence was recorded and combined. FIG. 4A shows weak fluorescence of few undifferentiated (vehicle-treated) cells, while FIG. 4B shows strong PPIX fluorescence in almost all cells.

### Example 6

This example shows increased PpIX formation in LNCaP tumors after R1881 treatment in vivo.

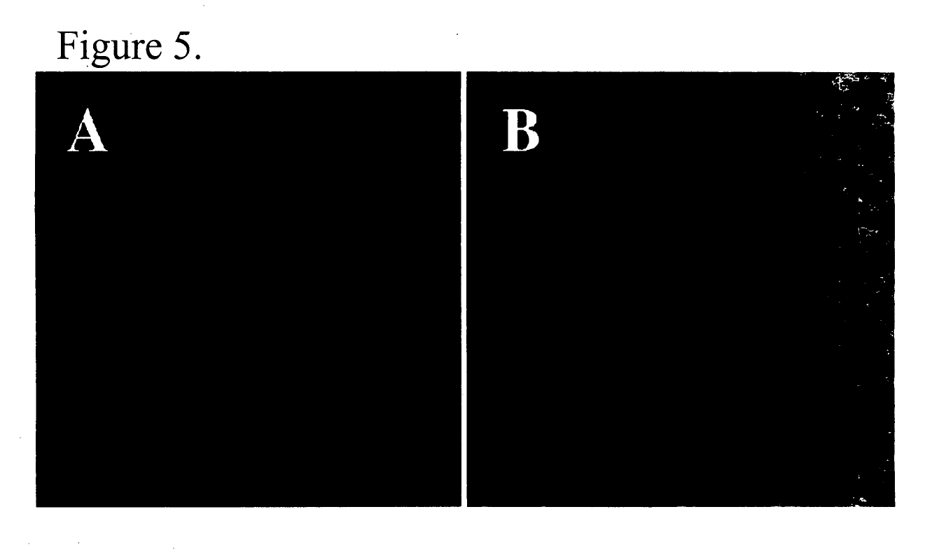

SCID mice bearing LNCaP tumors were pretreated with a high dose of 400 pg/kg R1881 and after 4 days received 250 mg/kg ALA i.p. At 4 hours, the mice were sacrificed and the tumors removed. Frozen sections of the tumors were imaged on the confocal laser scanning microscope. Using 633 nm excitation, the sections of tumors from an untreated mouse (FIG. 5A) and an R1881-pretreated mouse (FIG. 5B) were imaged at the same instrument parameters.

Although we recognize the limitations of quantitative fluorescence imaging, the confocal laser scanning microscope represents the best tool for such a comparison.

### Example 7

This example compares ALA-induced PpIX formation with and without pretreatment with chemotherapeutic agents, and comparing the ALA-dependent phototoxic effects with and without pretreatment with chemotherapeutic agents.

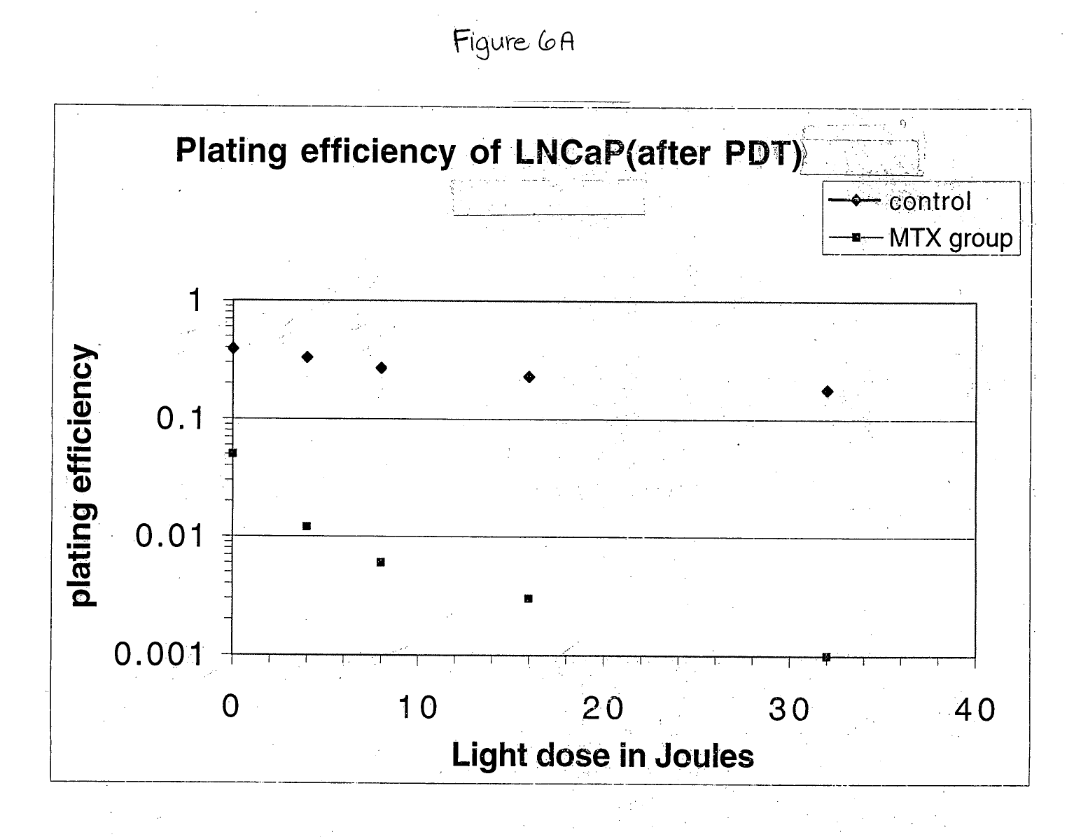

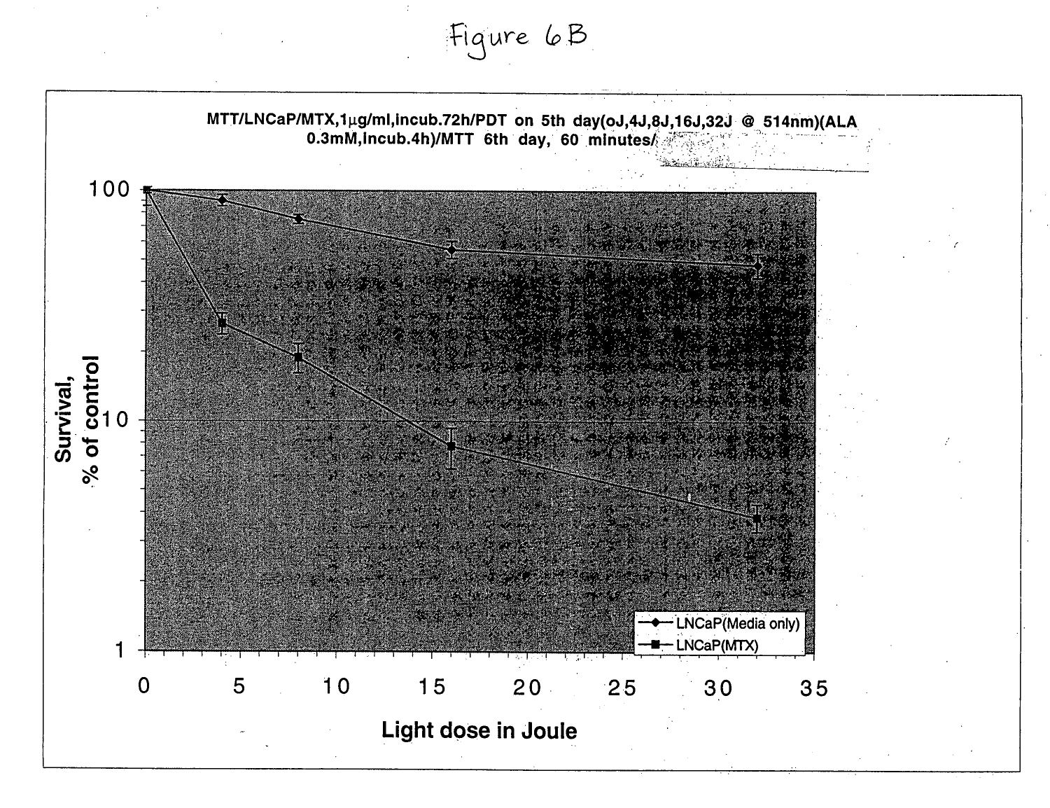

LNCaP (human prostate cancer) cells were treated with methotrexate (1 μg/ml) for 72 hours. The cells were then incubated with 0.3 mM ALA for 4 hours. After ALA treatment, cells were either used for quantification of PpIX or exposed to 514 nm light. The combined regimen resulted in 4.1±1.1-fold (mean ± standard deviation) increase in PpIX levels. Light-induced cytotoxicity as measured by MTT conversion assay is shown in FIG. 6A. While PDT alone required 20 J/cm2 to reduce cell survival by 50%, the combined regimen showed much higher sensitivity, requiring only 2 J/cm2 to reach this level of cytotoxicity. This enhancement of PDT by chemotherapy can also be seen when long-term survival was assessed by colony formation assay (FIG. 6B). Here, the 50% levels are reached at 25 J/cm2, compared to 2 J/cm2. These findings are supported by a similar methotrexate-induced increase of PpIX formation in the human ovarian cancer cell line OV5. Chemotherapeutic effect was also confirmed using cyclophosphamide. Together, these data demonstrate that PDT can be enhanced by specific pretreatment regimens using short-term exposure to chemotherapeutic agents.

What is claimed is:

1. A method of inhibiting unwanted cell proliferation, the method comprising the steps of: inducing differentiation in a cell with a chemotherapeutic agent; providing said cell with a photosensitizer; activating the photosensitizer; and inhibiting unwanted cell proliferation.

2. The method of claim 1, wherein the cell is malignant.

3. The method of claim 1, wherein a precursor of a photosensitizer is provided to the cell.

4. The method of claim 1, wherein the photosensitizer is chlorin e6 or a chlorin derivative.

5. The method of claim 1, wherein providing the cell with a photosensitizer comprises providing the cell with a compound which causes the accumulation of a photosensitizer, or a compound which causes the formation of a photosensitizer, or a compound that is converted to a photo sensitizer.

6. The method of claim 5, wherein the compound is a porphyrin, or a porphyrin precursor.

7. The method of claim 1, wherein the photosensitizer is coupled to a targeting moiety.

8. The method of claim 1, wherein the cell is benign.

9. The method of claim 2, wherein the cell is a malignant breast epithelial cell.

10. The method of claim 1, wherein the cell is a prostate cell.

11. The method of claim 10, wherein the prostate cell is a prostate carcinoma cell.

12. The method of claim 1, wherein cell is a tumor cell.

13. A method of treating a subject having a disorder characterized by unwanted cell proliferation, the method comprising the steps of:
(a) administering a chemotherapeutic agent to a subject in an amount sufficient to induce differentiation in the cell;
(b) administering a photosensitizer;
(c) activating the photosensitizer;
(d) inhibiting unwanted cell proliferation.

14. The method of claim 13, wherein the chemotherapeutic agent is selected from the group consisting of cyclophosphamide, ifosfamide, thiotepa, busulfan, carmustine, dabarcazine, methotrexate, 5-fluorouracil, 5-fluorodeoxyuridine, cytosine arabinoside (AraC), mercaptopurine, thioguanine, fludarabine phosphate, azathioprine, vinblastine, vincristine, vindesine, vinorelbine, paclitaxel, etoposide, teniposide, actinomycin D, daunorubicin, doxorubicin, idarubicin, bleomycin, mithramycin, mitomycin, cisplatin, carboplatin, and hydroxyurea.

15. The method of claim 13, wherein a photosensitizer is administered to the subject.

16. The method of claim 13, wherein administering the photosensitizer comprises administering a precursor of a photosensitizer.

17. The method of claim 13, wherein the photosensitizer is chlorin e6 or a chlorin derivative.

18. The method of claim 13, wherein administering a photosensitizer comprises administering a compound which causes the accumulation of a photosensitizer, or a compound which causes the formation of a photosensitizer, or a compound that is converted to a photo sensitizer.

19. The method of claim 13, wherein the photosensitizer is coupled to a targeting moiety.

20. The method of claim 13, wherein the subject has an unwanted proliferation of breast epithelial cells.

21. The method of claim 13, wherein the subject has an unwanted proliferation of prostate cells.

22. The method of claim 21, wherein the prostate cells are prostate carcinoma cells.

23. The method of claim 13, wherein the cell is induced to differentiate and the photosensitizer is provided such that the photosensitizer is present while the cells are in a state of induced differentiation.

24. The method of claim 13, wherein the chemotherapeutic agent is administered first.

25. The method of claim 13, wherein the photosensitizer is administered before the chemotherapeutic agent.

26. The method of claim 13, wherein the chemotherapeutic agent is administered before the photo sensitizer.

27. The method of claim 13, wherein the chemotherapeutic agent and the photosensitizer are concomitantly administered.

28. The method of claim 13, wherein the photosensitizer is activated before the chemotherapeutic agent is administered.

29. The method of claim 13, wherein the photosensitizer is activated after the chemotherapeutic agent is administered.
